(19) United States
(12) Patent Application Publication
(10) Pub. No.: US 2006/0105340 A1
(43) Pub. Date: May 18, 2006

(54) Novel tumor suppressor gene and compositions and methods for making and using the same

(76) Inventors: Carlo M. Croce, Columbus, OH (US); George A. Calin, Columbus, OH (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/530,792

(22) PCT Filed: Oct. 10, 2003
(86) PCT No.: PCT/US03/32270

§ 371 Date: May 13, 2005

(30) Foreign Application Priority Data
Oct. 11, 2002 (US) 60417842

(51) Int. Cl. C12Q 1/68; C07H 21/04; C12P 21/02; C12N 15/09; C07K 14/52

(52) U.S. Cl. 435/6; 435/69.5; 435/320.1; 435/325; 530/351; 536/23.5

(57) ABSTRACT

The present invention relates to the identification and cloning of ARTS-1, a novel tumor suppressor gene, to isolated proteins encoded by ARTS-1, and to methods of making and using the same.

### CROSS REFERENCE TO RELATED APPLICATIONS

This application claims the benefit of U.S. Provisional Application No. 60/417,842, filed Oct. 11, 2002, incorporated herein by reference in its entirety.

This invention was made with Government support under Program Project Grant P01CA76259, P01CA81534, and P30CA56036 from the National Cancer Institute. The Government has certain rights in this invention.

### FIELD OF THE INVENTION

The invention relates to the identification and cloning of ARTS1, a novel tumor suppressor gene, and to methods of making and using the same. ARTS1 was originally designated ARLTS1, and may also be referred to as ARLS1.

### BACKGROUND OF THE INVENTION

Loss-of-function mutations in tumor suppressor genes (TSGs) play an essential role in the initiation and progression of human tumors, while inactivation by methylation seems to be important for tumor progression (Weinberg, R. A. Tumor suppressor genes. Science 254, 1138-46. (1991), which is incorporated herein by reference). Chromosome 13 at band q14, where the retinoblastoma (RB1) gene (Marshall, C. J. Tumor suppressor genes. Cell 64, 313-26. (1991), which is incorporated herein by reference) is located, is hemizygously or homozygously deleted in a diversity of hematopoietic and solid tumors (Bullrich, F. & Croce, C. M. Molecular biology of chronic lymphocytic leukemia. In Chronic Lymphoid Leukemias (ed. Chenson, B. D.) 9-32 (Marcel Dekker, Inc., New York Bassel, 2001), which is incorporated herein by reference). Several reports presented evidence for a new tumor suppressor locus telomeric to the RB1 gene (Brown, A. G., Ross, F. M., Dunne, E. M., Steel, C. M. & Weir-Thompson, E. M. Evidence for a new tumour suppressor locus (DBM) in human B-cell neoplasia telomeric to the retinoblastoma gene. Nat Genet 3, 67-72. (1993), Howthorn, L. A., Chapman, R., Oscier, D. & Cowell, J. K. The consistent 13q14 translocation breakpoint seen in chronic B-cell leukaemia (BCLL) involves deletion of the D13S25 locus which lies distal to the retinoblastoma predisposition gene. Oncogene 8, 1415-9 (1993) and Liu, Y. et al. Chronic lymphocytic leukemia cells with allelic deletion at 13q14 commonly have one intact RBI gene: evidence for a role of an adjacent locus. Proc Natl Acad Sci USA 90, 8697-701. (1993) which are each incorporated herein by reference). However, none of the genes in the region were found to be inactivated by either combination of deletion, mutations or promoter hypermethylation.

There is a need to identify and clone TSGs whose loss of function are associated with initiation and progression of human tumors. There is a need to identify a TSG telomeric to the RBI gene. There is a need to identify nucleic acids which can serve as probes or primers for the detection of the TSG. There is a need for genetic based therapeutics which can be delivered to function in cells with a TSG mutation. There is a need for isolated protein and for antibodies which specifically react to the protein. There is a need for assays, reagents and kits to identify compounds that can upregulate, enhance or compensate for inactivity of the TSG. There is a need to study and understand the mechanisms by which the TSG is involved in initiation and progression of tumors and for reagents useful in such studies. There is a need to identify new cancer therapeutics and for kits and methods of identifying such compounds.

### SUMMARY OF THE INVENTION

The invention relates to isolated proteins comprising the amino acid sequence shown in SEQ ID NO:2.

The invention relates to isolated nucleic acid molecules that comprise nucleic acid sequences that encode a protein that has an amino acid sequence shown in SEQ ID NO:2.

The invention relates to isolated nucleic acid molecules that comprise SEQ ID NO:1 or a fragment thereof having at least 10 nucleotides.

The invention relates to a recombinant expression vector comprising the nucleic acid molecule comprising SEQ ID NO:1.

The invention relates to a host cell comprising a recombinant expression vector comprising the nucleic acid molecule that comprises SEQ ID NO:1.

The invention relates to an oligonucleotide molecule comprising a nucleotide sequence complimentary to a nucleotide sequence of at least 5 nucleotides of SEQ ID NO:1.

The invention relates to isolated antibodies that bind to an epitope on SEQ ID NO:2.

The invention relates to methods of identifying modulators of Caspase-1 protease activity.

### BRIEF DESCRIPTION OF THE DRAWINGS

FIG. 1. Localization of ARTS1 tumor suppressor gene at 13q14. A. The position of genetic markers and positions of genes on the map is shown. B. A multiple alignment of human ARTS1 (SEQ ID NO:2) with human ARL proteins. Several motifs presumably involved in nucleotide binding and hydrolysis (PM1, PM3, G2 and G3), characteristic of Ras-related GTPases27, are also present in ARTS1. Furthermore, five additional aminoacids typical of the ARF subfamily (G2, N47, W74, R95 and G161) are all conserved in ARTS1. In the C-terminus ARTS1 harbors less arginine or lysine residues than ARL4, ARL6 and ARL7. The location of Trp149Stop mutation is indicated by an arrow.

FIG. 2. ARTS1 mRNA expression and methylation analysis. A. Expression of ARTS1 by Northern blotting in cancer cell lines show absent or reduced expression in several cell lines. B. ARLTSG1 expression correlates with the level of methylation of this locus analyzed by Southern blotting of digested genomic DNA with Bg1II alone or in combination with HpaII. The combination Bg1II+MspI was used to determine the fragment length without respect of methylation. The presence or absence of ARTS1 expression is shown by “+” or “−”, respectively and the restriction map (Bg1II-B-thick vertical lines, HpaII-thin vertical lines) is drawn at the bottom. The position of the ORF probe used is indicated by *. C. Correlation between ARTS1 expression analyzed by RT-PCR and CpG sites methylation analyzed by bisulfite sequencing in fresh tumors; white and black rectangles represent unmethylated and hypermethylated CpGs respectively, while gray rectangles represent partially methylated CpG sites. As control we use Epstein-Barr Virus transformed lymphoblastoid cell lines.

FIG. 3. ARTS1 suppresses tumorigenicity and A549 cells. A. Restoration of ARTS1 expression by transfection of the minigene into A549. B. Tumor formation in nude mice. The weight (mg) of tumors for the five analyzed clones determined at the indicated times are shown. The same results were obtained by measurement of tumors. C. Example of tumorigenesis in nude mice at 8 weeks after s.c. injection of 10 6 cells. C. Colony growth in soft agar (data at 21 days after plating 5×10 4 cells).

FIG. 4. Analysis of ARTS1 expression in human tissues by Northern blotting reveals that ARTS1 is ubiquitously expressed.

FIG. 5. Mutation analysis in ARTS1 shows the presence of the germline polymorphism G446A (Trp149Stop). The presented sequences are in reverse orientation. For identification of the G446A (Trp149Stop) mutation a rapid assay was developed using the MaeI site introduced by the mutation. DNA was amplified using primers MaeI-F1 (which contains a changed base from the wild-type sequence to destroy a constitutive MaeI site) and MaeI-R1 (for sequences of the primers, see Table 4), purified using QIAquick PCR purification kit (QIAGEN) and digested with 2 U of MaeI (Boehringer Manhiem, Germany). The amplification of a normal allele gives rise to a single 138 bp product, while the mutant allele produces two bands of 106 and 32 bp. Note that the digestion has low efficiency and only partial digestion products were obtained. Digested PCR products were loaded on 3% agarose gel and visualized using a UV imager. N=normal and T=tumor.

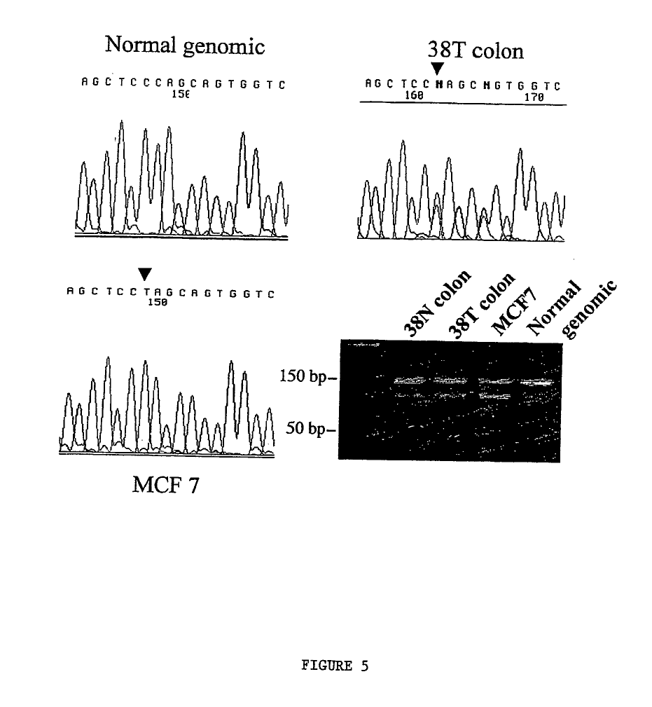

FIG. 6. Both wild-type ARTS1 and the truncated ÄCARLTS1 proteins are localized in cytoplasm and nucleus. Subcellular localization using ARTS1-GFP fusion protein 293 cells were transfected with pARLTS1-gfp, pÄC-ARLTS1-gfp and control plasmid, pEGFPN1. Bright field (right) and fluorescence images (left) of the same microscopy field are presented.

FIG. 7. The sequence of the cDNA of human ARTS1 (SEQ ID NO:1) is shown. The GenBank Accession number for the sequence is AF441378.

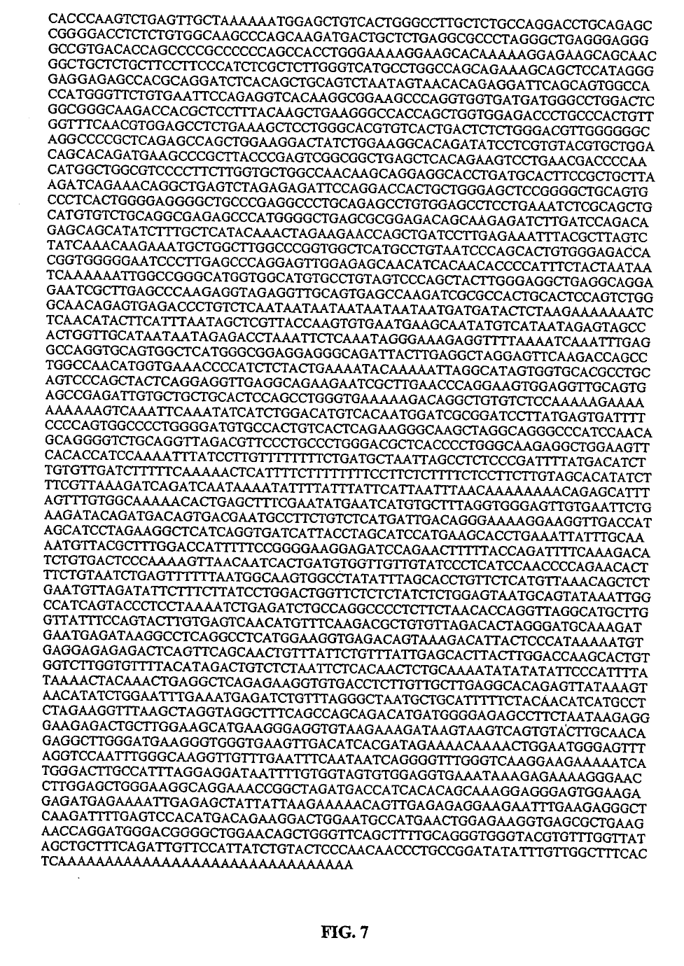

### DETAILED DESCRIPTION OF ILLUSTRATIVE EMBODIMENTS

The invention arises from the identification of ARTS1, a novel member of the ADP-ribosylation factor family. ARTS1 is located at 13q14, and displays features characteristic of a TSG. ARTS1 is downregulated by hypermethylation in 25 out of 75 (33%) human primary tumors and cell lines analyzed. Furthermore, analysis of 800 tumor and normal DNAs revealed the presence of several variants including a germline nonsense polymorphism G446A (Trp149Stop) that is three times more frequent in cancer patients with a family history of cancer than in the normal population. Restoration of wild-type ARTS1 expression in A549 cells, which shows low levels of expression, suppresses tumor formation of A549 cancer cells.

The GenBank accession number of the human ARTS1 cDNA is AF441378. During the final stages of the functional studies described below, a clone of 1.6 kb, BC013150, containing the ORF of ARTS1 and encoding the hypothetical protein FLJ22595 (accession number AAH13150) was deposited in the GenBank.

The ARTS1 gene, and proteins, polypeptides, or peptides encoded by the gene, can be used in methods of preventing abnormal cell growth in mammalian subjects. Such methods involve administering to a mammal a composition comprising an effective amount of the ARTS1 protein. Such methods also involve administering to a mammal a composition comprising an expression vector comprising a gene encoding ARTS1.

The discovery of ARTS1 provides the means to study its function as a TSG, to design probes and primers to detect its presence and/or to detect mutants, to prepare isolated nucleic acid molecules, to insert the nucleic acid molecules that encode ARTS1 into vectors such as cloning vectors to produce multiple copies, expression vectors useful to transform cells that will produce the protein and gene therapy vectors which can be used treat patients with tumors arising from a lack of endogenous ARTS1 function. Antisense compounds may be produced to generate tumor cells that lack ARTS1 function that can be used in assays to identify compounds useful to treat such cancers. Assays and kits can also be provided to identify compounds that upregulate or enhance ARTS1 activity. Transformed host cells may be used in methods to produce ARTS1 protein. Antibodies can be prepared that specifically bind to ARTS1 protein and used to isolate or detect the protein including to distinguish wild type from mutants.

In certain embodiments, the present invention provides isolated ARTS1 protein that comprises the amino acid sequence shown in SEQ ID NO:2. The ARTS1 protein can be isolated from natural sources, produced by recombinant DNA methods or synthesized by standard protein synthesis techniques. In other embodiments, the invention relates to ARTS1-like polypeptides, which are polypeptides that are similar to, but differ from, the ARTS1 polypeptide by having at least one amino acid substitution or deletion. For example, conservative amino acid substitutions may be made at one or more nonessential amino acid residues of the ARTS1 protein to generate ARTS1-like polypeptides. A “nonessential” amino acid residue is a residue that can be altered from the wild-type sequence of ARTS1 protein (e.g., the sequence of SEQ ID NO:2) without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Such substitutions would not be made for conserved amino acid residues, or for amino acid residues residing within a conserved motif.

Antibodies which specifically bind to the ARTS1 protein may be used to purify the protein from natural sources using well known techniques and readily available starting materials. Such antibodies may also be used to purify ARTS1 from material present when producing the protein by recombinant DNA methodology. As used herein, the term “antibody” is meant to refer to complete, intact antibodies, and fragments including Fab fragments and F(ab)2 fragments. Complete, intact antibodies include monoclonal antibodies such as murine monoclonal antibodies, chimeric antibodies, primatized antibodies and humanized antibodies. Antibodies that bind to an epitope present on ARTS1 are useful to isolate and purify ARTS1 from both natural sources or recombinant expression systems using well known techniques such as affinity chromatography. Such antibodies are useful to detect the presence of such protein in a sample and to determine if cells are expressing the protein.

The production of antibodies and the protein structures of complete, intact antibodies, and fragments such as Fab fragments and F(ab)2 fragments and the organization of the genetic sequences that encode such molecules are well known and are described, for example, in Harlow, E. and D. Lane (1988) ANTIBODIES: A Laboratory Manual, Cold Spring Harbor Laboratory, Cold Spring Harbor, N.Y. which is incorporated herein by reference. Briefly, for example, ARTS1 or an immunogenic fragment thereof is injected into mice. The spleen of the mouse is removed, the spleen cells are isolated and fused with immortalized mouse cells. The hybrid cells, or hybridomas, are cultured and those cells that secrete antibodies are selected. The antibodies are analyzed and, if found to specifically bind to ARTS1, the hybridoma which produces them is cultured to produce a continuous supply of antibodies.

According to some embodiments, the present invention relates to an isolated nucleic acid molecule comprising a nucleotide sequence that encodes the amino acid sequence of SEQ ID NO:2. Such molecules can be routinely designed using the information set forth in SEQ ID NO:2. In certain embodiments, the invention relates to an isolated nucleic acid molecule comprising SEQ ID NO:1. Nucleic acid molecules that are fragments of nucleic acid molecules comprising a nucleotide sequence that encode the amino acid sequence of SEQ ID NO:2 and of nucleic acid molecules comprising SEQ ID NO:1 are also encompassed by the present invention. By “fragment” is intended a portion of the nucleotide sequence encoding the ARTS1 protein or an ARTS1-like polypeptide. A fragment of an ARTS1 nucleotide sequence may encode a biologically active portion of an ARTS1-like protein, or it may be a fragment that can be used as a hybridization probe or PCR primer. A biologically active portion of an ARTS1-like protein can be prepared by isolating a portion of one of the nucleotide sequences of the invention, expressing the encoded portion of the ARTS1-like protein (e.g., by recombinant expression in vitro), and assessing the activity of the encoded portion of the ARTS1-like protein. Nucleic acid molecules that are fragments of an ARTS1-like nucleotide sequence comprise at least about 10, 15, 20, 50, 75, 100, 200, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950 nucleotides, or up to the number of nucleotides present in a full-length ARTS1-like nucleotide sequence disclosed herein (for example, up to 3791 nucleotides for SEQ ID NO:1), depending upon the intended use. Nucleic acid molecules that are variants of the ARTS1 nucleotide sequences disclosed herein are also encompassed by the present invention. “Variants” of the ARTS1 nucleotide sequences include those sequences that encode the ARTS1 protein or ARTS1-like polypeptides disclosed herein but that differ conservatively because of the degeneracy of the genetic code. These naturally occurring allelic variants can be identified with the use of well-known molecular biology techniques, such as polymerase chain reaction (PCR) and hybridization techniques as outlined below. Variant nucleotide sequences also include synthetically derived nucleotide sequences that have been generated, for example, by using site-directed mutagenesis but which still encode the ARTS1-like proteins. Generally, nucleotide sequence variants of the invention will have at least about 45%, 55%, 65%, 70%, 75%, 80%, 85%, 90%, 91%, 92%, 93%, 94%, 95%, 96%, 97%, 98%, or 99% identity to SEQ ID NO:1.

Using standard techniques and readily available starting materials, a nucleic acid molecule that encodes ARTS1 may be isolated from a cDNA library, using probes or primers which are designed using the nucleotide sequence information disclosed in SEQ ID NO:1. In some embodiments, the nucleic acid molecules comprise the nucleotide sequence that consists of the coding sequence in SEQ ID NO:1. In some embodiments, the nucleic acid molecules consist of the nucleotide sequence set forth in SEQ ID NO:1. The isolated nucleic acid molecules of the invention are useful to prepare constructs and recombinant expression systems for preparing ARTS1.

A cDNA library may be generated by well-known techniques. A cDNA clone which contains one of the nucleotide sequences set out is identified using-probes that comprise at least a portion of the nucleotide sequence disclosed in SEQ ID NO:1. The probes have at least 16 nucleotides, preferably 24 nucleotides. The probes are used to screen the cDNA library using standard hybridization techniques. Alternatively, genomic clones may be isolated using genomic DNA from any human cell as a starting material. In certain embodiments, the present invention relates to isolated nucleic acid molecules that comprise a nucleotide sequence identical or complementary to a fragment of SEQ ID NO:1 which is at least 10 nucleotides. In some embodiments, the isolated nucleic acid molecules consist of a nucleotide sequence identical or complementary to a fragment of SEQ ID NO:1 which is at least 10 nucleotides. In some embodiments, the isolated nucleic acid molecules comprise or consist of a nucleotide sequence identical or complementary to a fragment of SEQ ID NO:1 which is 15-150 nucleotides. In some embodiments, the isolated nucleic acid molecules comprise or consist of a nucleotide sequence identical or complementary to a fragment of SEQ ID NO:1 which is 15-30 nucleotides. Isolated nucleic acid molecules that comprise or consist of a nucleotide sequence identical or complementary to a fragment of SEQ ID NO:1 which is at least 10 nucleotides are useful as probes for identifying genes and cDNA sequence having SEQ ID NO:1, PCR primers for amplifying genes and cDNA having SEQ ID NO:1, and antisense molecules for inhibiting transcription and translation of genes and cDNA, respectively, which encode ARTS1 having the amino acid sequence of SEQ ID NO:2.

The cDNA that encodes ARTS1 may be used as a molecular marker in electrophoresis assays in which cDNA from a sample is separated on an electrophoresis gel and ARTS1 probes are used to identify bands which hybridize to such probes. Specifically, SEQ ID NO:1 or portions thereof, may be used as a molecular marker in electrophoresis assays in which cDNA from a sample is separated on an electrophoresis gel and ARTS1 specific probes are used to identify bands which hybridize to them, indicating that the band has a nucleotide sequence complementary to the sequence of the probes. The isolated nucleic acid molecule provided as a size marker will show up as a positive band that is known to hybridize to the probes and thus can be used as a reference point to the size of cDNA that encodes ARTS1. Electrophoresis gels useful in such an assay include standard polyacrylamide gels as described in Sambrook et al., Molecular Cloning a Laboratory Manual, Second Ed. Cold Spring Harbor Press (1989) which is incorporated herein by reference.

The nucleotide sequences in SEQ ID NO:1 may be used to design probes, primers and complementary molecules which specifically hybridize to the unique nucleotide sequences of ARTS1. Probes, primers and complementary molecules which specifically hybridize to nucleotide sequence that encodes ARTS1 may be designed routinely by those having ordinary skill in the art. As used herein, the term “specifically hybridize to nucleotide sequence that encodes ARTS1” is meant to refer to nucleic acid molecules with unique nucleotide sequences that hybridize to ARTS1 encoding sequences but not other known protein encoding sequences, such as sequences identical to portions of SEQ ID NO:1. This, the unique sequences described herein are those that do not overlap with known sequences.

The present invention also includes labeled oligonucleotides that are useful as probes for performing oligonucleotide hybridization methods to identify ARTS1. The oligonucleotides include sequences that specifically hybridize to nucleotide sequences that encode ARTS1. Accordingly, the present invention includes probes that can be labeled and hybridized to unique nucleotide sequences that encode ARTS1. The labeled probes of the present invention are labeled with radiolabelled nucleotides or are otherwise detectable by readily available nonradioactive detection systems. In some preferred embodiments, probes comprise oligonucleotides consisting from 10 to 100 nucleotides. In some preferred embodiments, probes comprise oligonucleotides consisting of from 10 to 50 nucleotides. In some preferred embodiments, probes comprise oligonucleotides consisting of from 12 to 20 nucleotides. The probes preferably contain nucleotide sequence completely identical or complementary to a fragment of a unique nucleotide sequences of ARTS1.

PCR technology is practiced routinely by those having ordinary skill in the art and its uses in diagnostics are well known and accepted. Methods for practicing PCR technology are disclosed in “PCR Protocols: A Guide to Methods and Applications”, Innis, M. A., et al. Eds. Academic Press, Inc. San Diego, Calif. (1990) which is incorporated herein by reference. Applications of PCR technology are disclosed in “Polymerase Chain Reaction” Erlich, H. A., et al., Eds. Cold Spring Harbor Press, Cold Spring Harbor, N.Y. (1989) which is incorporated herein by reference. Some simple rules aid in the design of efficient primers. Typical primers are 18-28 nucleotides in length having 50% to 60% g+c composition. The entire primer is preferably complementary to the sequence it must hybridize to. Preferably, primers generate PCR products having from 100 base pairs to 2000 base pairs. However, it is possible to generate products of 50 base pairs to up to 10 kb and more.

PCR technology allows for the rapid generation of multiple copies of nucleotide sequences by providing 5′ and 3′ primers that hybridize to sequences present in a nucleic acid molecule, and further providing free nucleotides and an enzyme which fills in the complementary bases to the nucleotide sequence between the primers with the free nucleotides to produce a complementary strand of DNA. The enzyme will fill in the complementary sequences adjacent to the primers. If both the 5′ primer and 3′ primer hybridize to nucleotide sequences on the complementary strands of the same fragment of nucleic acid, exponential amplification of a specific double-stranded product results. If only a single primer hybridizes to the nucleic acid molecule, linear amplification produces single-stranded products of variable length. PCR primers include at least one primer which includes a nucleotide sequence that specifically hybridizes to nucleotide sequence that encodes ARTS1.

One having ordinary skill in the art can isolate the nucleic acid molecule that encode ARTS1 and insert it into an expression vector using standard techniques and readily available starting materials.

The present invention relates to a recombinant expression vector that comprises a nucleotide sequence that encodes ARTS1 that comprises the amino acid sequence of SEQ ID NO:2. As used herein, the term “recombinant expression vector” is meant to refer to a plasmid, phage, viral particle or other vector which, when introduced into an appropriate host, contains the necessary genetic elements to direct expression of the coding sequence that encodes the ARTS1 of the invention. The coding sequence is operably linked to the necessary regulatory sequences. Expression vectors are well known and readily available. Examples of expression vectors include plasmids, phages, viral vectors and other nucleic acid molecules or nucleic acid molecule containing vehicles useful to transform host cells and facilitate expression of coding sequences. In some embodiments, the recombinant expression vector comprises the nucleotide sequence set forth in SEQ ID NO:1. The recombinant expression vectors of the invention are useful for transforming hosts to prepare recombinant expression systems for preparing ARTS1.

The present invention relates to a host cell that comprises the recombinant expression vector that includes a nucleotide sequence that encodes ARTS1 that comprises SEQ ID NO:1. In some embodiments, the host cell comprises a recombinant expression vector that comprises SEQ ID NO:1. Host cells for use in well known recombinant expression systems for production of proteins are well known and readily available. Examples of host cells include bacteria cells such as E. coli, yeast cells such as S. cerevisiae, insect cells such as S. frugiperda, non-human mammalian tissue culture cells chinese hamster ovary (CHO) cells and human tissue culture cells such as HeLa cells.

The present invention relates to a transgenic non-human mammal that comprises the recombinant expression vector that comprises a nucleic acid sequence that encodes ARTS1 that comprises the amino acid sequence of SEQ ID NO:2. Transgenic non-human mammals useful to produce recombinant proteins are well known as are the expression vectors necessary and the techniques for generating transgenic animals. Generally, the transgenic animal comprises a recombinant expression vector in which the nucleotide sequence that encodes ARTS1 is operably linked to a mammary cell specific promoter whereby the coding sequence is only expressed in mammary cells and the recombinant protein so expressed is recovered from the animal's milk. In some embodiments, the coding sequence that encodes ARTS1 is SEQ ID NO:1.

In some embodiments, for example, one having ordinary skill in the art can, using well known techniques, insert such DNA molecules into a commercially available expression vector for use in well known expression systems. For example, the commercially available plasmid pSE420 (Invitrogen, San Diego, Calif.) may be used for production of collagen in E. coli. The commercially available plasmid pYES2 (Invitrogen, San Diego, Calif.) may, for example, be used for production in S. cerevisiae strains of yeast. The commercially available MAXBAC™ complete baculovirus expression system (Invitrogen, San Diego, Calif.) may, for example, be used for production in insect cells. The commercially available plasmid pcDNA I (Invitrogen, San Diego, Calif.) may, for example, be used for production in mammalian cells such as Chinese Hamster Ovary cells. One having ordinary skill in the art can use these commercial expression vectors and systems or others to produce Caspase-1 using routine techniques and readily available starting materials. (See e.g., Sambrook et al., Molecular Cloning a Laboratory Manual, Second Ed. Cold Spring Harbor Press (1989) which is incorporated herein by reference.) Thus, the desired proteins can be prepared in both prokaryotic and eukaryotic systems, resulting in a spectrum of processed forms of the protein.

One having ordinary skill in the art may use other commercially available expression vectors and systems or produce vectors using well known methods and readily available starting materials. Expression systems containing the requisite control sequences, such as promoters and polyadenylation signals, and preferably enhancers, are readily available and known in the art for a variety of hosts. See e.g., Sambrook et al., Molecular Cloning a Laboratory Manual, Second Ed. Cold Spring Harbor Press (1989).

A wide variety of eukaryotic hosts are also now available for production of recombinant foreign proteins. As in bacteria, eukaryotic hosts may be transformed with expression systems which produce the desired protein directly, but more commonly signal sequences are provided to effect the secretion of the protein. Eukaryotic systems have the additional advantage that they are able to process introns which may occur in the genomic sequences encoding proteins of higher organisms. Eukaryotic systems also provide a variety of processing mechanisms which result in, for example, glycosylation, carboxy-terminal amidation, oxidation or derivatization of certain amino acid residues, conformational control, and so forth.

Commonly used eukaryotic systems include, but are not limited to, yeast, fungal cells, insect cells, mammalian cells, avian cells, and cells of higher plants. Suitable promoters are available which are compatible and operable for use in each of these host types as well as are termination sequences and enhancers, e.g. the baculovirus polyhedron promoter. As above, promoters can be either constitutive or inducible. For example, in mammalian systems, the mouse metallothionein promoter can be induced by the addition of heavy metal ions.

The particulars for the construction of expression systems suitable for desired hosts are known to those in the art. Briefly, for recombinant production of the protein, the DNA encoding the polypeptide is suitably ligated into the expression vector of choice. The DNA is operably linked to all regulatory elements which are necessary for expression of the DNA in the selected host. One having ordinary skill in the art can, using well known techniques, prepare expression vectors for recombinant production of the polypeptide.

The expression vector including the DNA that encodes the Caspase-1 is used to transform the compatible host that is then cultured and maintained under conditions wherein expression of the foreign DNA takes place. The protein of the present invention thus produced is recovered from the culture, either by lysing the cells or from the culture medium as appropriate and known to those in the art. One having ordinary skill in the art can, using well known techniques, isolate ARTS1 that is produced using such expression systems. The methods of purifying ARTS1 from natural sources using antibodies which specifically bind to ARTS1 as described above, may be equally applied to purifying ARTS1 produced by recombinant DNA methodology.

Examples of genetic constructs include ARTS1 coding sequence operably linked to a promoter that is functional in the cell line into which the constructs are transfected. Examples of constitutive promoters include promoters from cytomegalovirus or SV40. Examples of inducible promoters include mouse mammary leukemia virus or metallothionein promoters. Those having ordinary skill in the art can readily produce genetic constructs useful for transfecting with cells with DNA that encodes ARTS1 from readily available starting materials. Such gene constructs are useful for the production of ARTS1.

In some embodiments of the invention, transgenic non-human animals are generated. The transgenic animals according to the invention contain SEQ ID NO:1 under the regulatory control of a mammary specific promoter. One having ordinary skill in the art using standard techniques, such as those taught in U.S. Pat. No. 4,873,191 issued Oct. 10, 1989 to Wagner and U.S. Pat. No. 4,736,866 issued Apr. 12, 1988 to Leder, both of which are incorporated herein by reference, can produce transgenic animals which produce ARTS1. Preferred animals are rodents, particularly rats and mice, and goats.

In addition to producing these proteins by recombinant techniques, automated peptide synthesizers may also be employed to produce ARTS1. Such techniques are well known to those having ordinary skill in the art and are useful if derivatives that have substitutions not provided for in DNA-encoded protein production.

One aspect of the invention relates to gene therapy, specifically “gene replacement.” Gene replacement” refers to the replacement of a mutated genetic element with a normal gene. The present invention provides methods of gene therapy that is a “gene replacement” therapy. Generally the present gene replacement method involves inhibition of an abnormal ARTS-1 product coupled with replacement with the normal ARTS-1 gene. Generally, methods of the present invention can be used to treat conditions associated with tumorogenesis related to a lack of or insufficient amount of functional wild type ARTS-1. Methods of the present invention may be used to replace the abnormal ARTS-1 gene with a normal ARTS-1 gene.

By normal ARTS-1 gene is meant any gene which, when encoded produces a biologically active, wild-type tumor suppressing ARTS-1 protein. By abnormal or mutant gene is meant any gene which, when encoded, does not produce a biologically active, wild-type ARTS-1 protein and/or is insufficiently present to perform a tumor suppression function.

The term “DNA construct” as used herein refers to any DNA molecule which has been modified such that the nucleotide sequences in the molecule are not identical to a sequence which is produced naturally.

The term “expression vector”, as used herein, is defined as a DNA construct which includes an autonomous site of replication, a site of transcription initiation, and at least one structural gene coding for a protein which is to be expressed in a host organism. The expression vector will usually also contain appropriate control regions such as a promoter and terminator which control the expression of the protein in the host organism. Expression vectors of the present invention may include retroviral vectors such as the “double copy” vector. As one skilled in the art would recognize, the particular vector chosen depends partly upon the cell-type targeted.

In preferred embodiments of the present invention the expression vector includes a promoter. Vectors encoding one or more ribozymes should preferably utilize a strong, RNA polymerase III type promoter. Useful promoters include, but are not limited to tRNA and SV40 promoters. Expression vectors of the present invention may also include homologous sequences with a host gene to provide for integration of the modified gene into the chromosome of the host.

The term “bifunctional expression vector” as used herein is defined as an expression vector which contains at least one structural gene cassette coding for a protein which is to be expressed in a host organism and a regulatory cassette coding for a regulatory element. The regulatory cassette may code for any element which functions within the cell to inhibit the expression of one or more genes. In accordance with preferred embodiments of the present invention the regulatory cassette codes for an RNA fragment having ribozyme activity effective to cleave a separate RNA molecule.

Cassette, as used herein, refers to a discrete DNA fragment that encodes a control region and a DNA sequence of interest such a structural protein.

The term “plasmid” is used herein in accordance with its commonly accepted meaning, i.e. autonomously replicating, usually close looped, DNA.

“Ribozyme” as the term is used herein, refers to an enzyme which is made of RNA. Ribozymes are involved in the cleavage and/or ligation of RNA chains. In preferred embodiments of the present invention, “hammerhead ribozymes” are used. As described above, hammerhead ribozymes cleave the phosphodiester bond of a target RNA downstream of a GUX triplet where X can be C, U, or A. Hammerhead ribozymes used in methods of the present invention have a structural domain having the sequence 3′-CAAAGCAGGAGCGCCUGAGUAGUC-5′ (SEQ ID NO:3). Site specific regulatory elements such as site specific ribozymes are provided in accordance with the present invention. The ribozyme regulatory element is made site specific, having the sequence 3′-Xn-CAAAGCAGGAGCGCCUGAGUAGUC-Ym-5′ ((SEQ ID NO:4), reported in 5′ to 3′ direction) where X and Y are complementary to regions of the target mRNA flanking the GUC site and n+m are generally from about 20 to about 35 RNA bases in length. n+m need not be of equal lengths although it is preferable that neither n nor m is less than about 10.

Hammerhead ribozymes target the triplet GUC. For a gene of interest a target site can be identified by analyzing the gene sequence to identify GUC triplets. Computer analysis of secondary structure may assist in site selection. Denman, (1993), Biotechniques, 15, 1090-1094.

Vectors of the present invention may be delivered to a patient via methods known in the art. Retroviral mediated delivery is particularly preferred in some embodiments of the invention. In vivo delivery by of retroviral vectors may be achieved, for example by i.v. injection of the retroviral vectors. A double balloon catheter may also be used for direct delivery of retroviral vectors to the patient.

According to one aspect of the invention, compounds may be screened to identify compounds that inhibit or enhance Caspase-1 activity. Substrates of Caspase-1 include baculovirus protein p35 and the Sf immunophillin FKBP46. Assays may be performed combining Caspase-1 with a substrate in the presence or absence of a test compound. The level of Caspase-1 activity in the presence of the test compound is compared to the level in the absence of the test compound. If Caspase-1 activity is increased by the presence of the test compound, the test compound is an enhancer. If Caspase-1 activity is decreased by the presence of the test compound, the test compound is an inhibitor. In some embodiments of the invention, the preferred concentration of test compound is from 1 μM to 500 μM. A preferred concentration is from 10 μM to 100 μM. In some preferred embodiments, it is desirable to use a series of dilutions of test compounds.

Kits are included which comprise containers with reagents necessary to screen test compounds. Such kits include a container with Caspase-1 protein, a container with a substrate such as FKBP46 or p35, which is preferably a labeled substrate, and instructions for performing the assay. Kits may include a control inhibitor such as anti-Caspase-1 neutralizing antibodies.

Combinatorial libraries may be screened to identify compounds that enhance or inhibit Caspase-1 activity.

### EXAMPLES

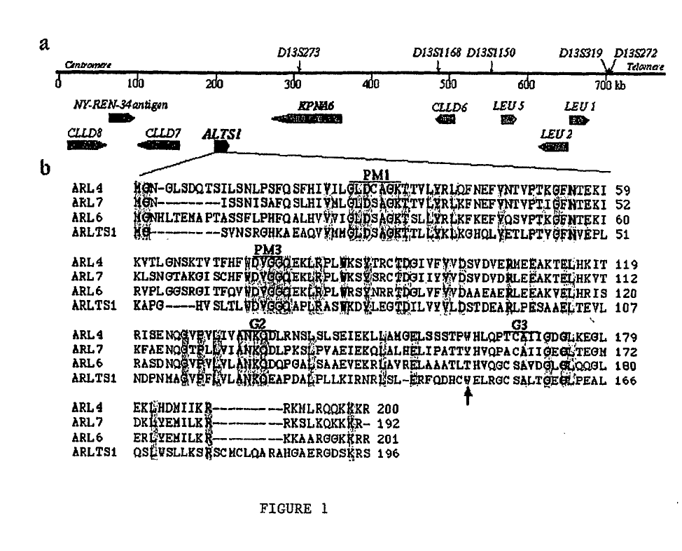

EXOFISH (Roest Crollius, et al. Estimate of human gene number provided by genome-wide analysis using Tetaodon nigroviridis DNA sequence. Nat Genet 25, 235-8. (2000), which is incorporated herein by reference) was used to scan 1.4 Mb of assembled genomic sequence at chromosome 13q14 (Mabuchi, H. et al. Cloning and characterization of CLLD6, CLLD7, and CLLD8, novel candidate genes for leukemogenesis at chromosome 13q14, a region commonly deleted B-cell chronic lymphocytic leukemia. Cancer Res 61, 2870-7. (2001), Bullrich, F. et al. Characterization of the 13q14 tumor suppressor locus in CLL: identification of ALT1, an alternative splice variant of the LEU2 gene. Cancer Res 61. 6640-8, (2001), Lander, E. S. et al. Initial sequencing and analysis of the human genome. Nature 409, 860-921. (2001), and Venter, J. C. et al. The sequence of the human genome. Science 291, 1304-51. (2001) which are each incorporated herein by reference) for putative genes. A 182 bp ‘ecore’ (evoluntionary conserved region) coding for an aminoacidic sequence with high homology to several members of the ADP-ribosylation factor family was found. By using EST walking and RACE, the corresponding full-length cDNA was obtained. Comparison with the genomic sequence indicated that the cloned cDNA, which was designated ARTS1 (for ADP-Ribosylation factor-Like, putative Tumor Suppressor gene 1), derives from a small gene composed of two exons separated by a 1.8 kb intronic sequence and spanning about 6 kb of DNA. Using LOH analysis it was found that this region was heterozygously deleted in a fraction of tumors between 10% (colon cancers) and 20% (B-CLL). The putative ORF, within the second exon, encodes a 196-amino acid protein with a predicted molecular mass of 21 kDa. BLAST analysis and Conserved Domain search of protein databases revealed highly significant homology with the ADP-ribosylation factor (ARF) and ARF-like (ARL) protein subfamily of the ras family (Moss, J. & Vaughan, M. Molecules in the ARF orbit. J Biol Chem 273, 21431-4. (1998), and Kahn, R. A., Der, C. J. & Bokoch, G. M. The ras superfamily of GTP-binding proteins: guidelines of nomenclature. Faseb J 6, 2512-3 (1992), which are each incorporated herein by reference). At the protein level, related proteins share at most 45% identical amino acids. A multiple alignment with the CLUSTALW program indicates that ARTS1 belongs to the subgroup formed by ARL4, ARL6 and ARL7 (Jacobs, S. et 1. ADP-ribosylation factor (ARF)-like 4, 6, and 7 represent a subgroup of the ARF family characterized by rapid nucleotide exchange and nuclear localization singnal. FEBS Lett 456, 384-8. (1999)) (FIG. 1).

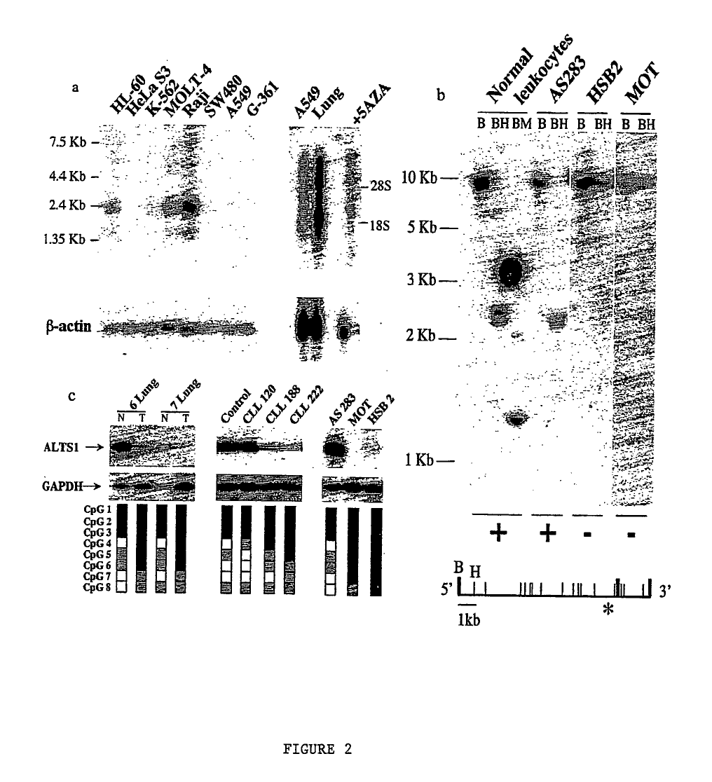

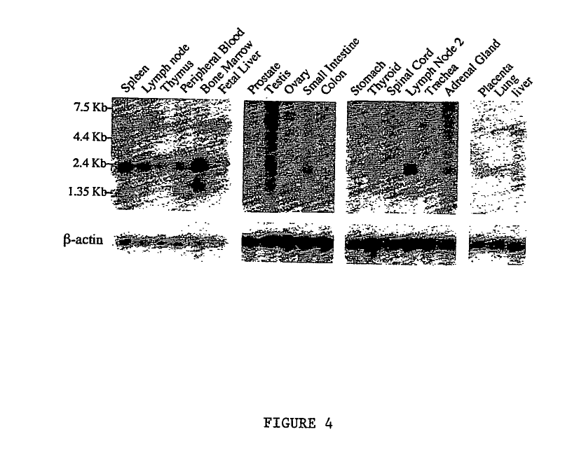

Northern analysis of normal human tissues with an ARTS1 probe revealed ubiquitous expression of a 2.2 kb transcript. In some tissues, two additional minor bands of approximately 1.3 and 5.5 kb were detected resulting from the use of different polyadenylation sites (FIG. 4). The expression of ARTS1 was analyzed by Northern blot and/or semiquantitative RT-PCR in a set of 59 hematopoietic and solid tumor cell lines. ARTS1 expression was significantly reduced or absent in 22% ( 7/32) of blood cancer cell lines, 78% ( 7/9) of lung cancer cell lines, 33% ( 2/6) of esophageal cancer cell lines and 22% of pancreatic cancer cell lines as well as in HeLa S3 (cervical carcinoma), SW 480 (colorectal cancer) and G-361 (melanoma) cell lines. In addition, 4 out of 16 fresh tumor samples (25%, 2/7 lung carcinomas and 2/9 B-CLL) for which cDNA and/or RNA were available showed reduction or absence of ARTS1 expression when compared to their normal tissue counterparts (FIG. 2 and Table 5).

The possibility that, as occurs with other cancer-related genes such as TSCL1 (Kuramochi, M. et al. TSLC1 is a tumor-suppressor gene in human non-small-cell lung cancer. Nat Genet 27, 427-30. (2001) which is incorporated herein by reference) or p16 (Merlo, A. et al. 5′ CpG island methylation is associated with transcriptional silencing of the tumour suppressor p16/CDKN2/MTS1 in human cancers. Nat Med 1, 686-92. (1995) which is incorporated herein by reference), ARTS1 is downregulated through hypermethylation of the putative promoter was examined. First, the global methylation level around ARTS1 was analyzed by Southern blotting using cell lines for which expression data was available. The level of expression is correlated with the methylation status of the genomic region—cell lines with low or no ARTS1 expression are highly methylated, while cell lines with normal levels of expression display only one methylated site (FIG. 2). ARTS1 DNA methylation patterns were examined in more detail through bisulfite sequencing to determine the methylation status of 5 CpG sites near the putative promoter sequences. Fresh tumor samples and tumor cell lines with low or absent ARTS1 expression showed higher methylation levels than normal tissues or tumors with normal expression levels (FIG. 2 and Table 5).

During an initial mutation screening with 80 cell lines (including 70 used for gene expression and 10 melanoma cell lines), three mutations have been identified. The first, a missense mutation G446A (Trp149Stop) is present in homozygosity in the MCF7 breast cell line and in heterozygosity in the HS776T pancreatic carcinoma cell line. Two heterozygous substitutions were identified in melanoma cell lines: a T50C (Met17Thr) and a C262A (Leu88Met) in one of the patients with T50C (Table 1).

In order to establish the significance of these mutations three panels of samples were screened (Methods and Table 6). The first includes 216 human tumors that were screened by direct sequencing of the ARTS1 ORF. Eight cases carried the G446A (Trp149Stop) mutation, including 3 breast cancers ( 3/48 of cases, 6.25%), 2 colorectal carcinomas ( 2/58, 3.45%), 1 lung carcinoma (⅕, 20%), 1 thyroid tumor ( 1/65, 1.5%) and one idiopathyc pancytopenia. All tumor samples had both the wild-type and mutant alleles except for a breast tumor with LOH at the ARTS1 locus, which was homozygous for the mutation. Sequencing of the ARTS1 in paired normal tissues, which were available for three out of six tumors, revealed the same alteration in the germline of patients.

The second panel contains 109 blood DNAs from patients with multiple cancers or with a family history of cancer screened by direct sequencing. Six additional cases with the G446A (Trp149Stop) were identified—2 malignant melanomas+prostate carcinoma cases ( 2/17, 11.75%), 2 cases of familial CLL ( 2/17, 11.75%), 1 case of pancreatic+melanoma (⅙, 16.5%) and 1 breast cancer ( 1/69 of cases, 1.5%) (see Table 2 for family history). At the protein level, the stop codon inserts a premature termination 48 amino acids before the C-terminus, leading to the synthesis of a smaller protein with 148 instead of 196 amino acids (FIG. 1). Thus, the truncated protein lacks the C-terminus motif presumably involved in nucleotide binding and hydrolysis characteristic of Ras related GTPases, one of the five additional amino acids typical of the ARF subfamily (Gly161) and the putative nuclear localization signal. Furthermore, Trp149, the site of the mutation is conserved in ARL4 and in 11 other ARF or ARF related genes including all six ARF genes.

The third panel comprises the case-controls: allele frequency for the G446A (Trp149Stop) mutation in three separate Caucasian cohorts was 2.10%, with variations between 0.86% ( 1/116) in the US population and 3.44% ( 7/203) in the Italian population. Overall, 14 patients out of 325 analyzed (4.63%) and 10 out of 475 normal controls (2.1%) had the stop mutation. The odds of G446A (Trp149Stop) were 2.10 (95% CI 0.92-4.77) times higher in cancer patients versus controls. After stratification upon family history of cancer, this odds increase in the group with positive family history to 2.70 (95% CI 0.85-8.32) (Table 6). In addition to the G446A (Trp149Stop), several other variants in the ARTS1 gene were identified including a G490A (Glu164Lys) substitution in a thyroid adenoma (Table 1). Four mutations in a total of 64 thyroid adenomas and carcinomas analyzed were found (two C65T missenses, one G446A nonsense and one G490A missense). All four mutations were found in adenomas of follicular origin, whereas all samples of non-follicular hystotype ( 42/65, 65%) were wild-type. It is highly unlikely that this allelic distribution is random (P=0.01 at Fisher exact test). Also, a G446A homozygous patient in a family with CLL has thyroid adenoma (Table 2). Taken together, these observations raise the possibility that this ARTS1 is involved in a portion of thyroid tumors with follicular histotype.

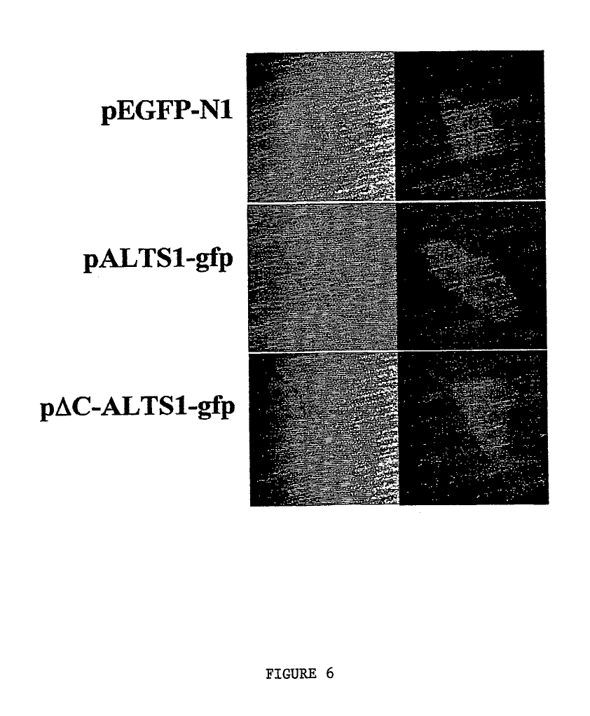

ARTS1 appears to be the first ARF family member reported to be altered in human cancers. Because of their nuclear localization signal (NLS), ARL4, ARL6 and ARL7 appear to be cargo molecules transported via the translocators importin-á and â in the nucleus where they have yet unknown functions. Of note, ARTS1 lacks a classical NLS at its C-terminus, and probably contains an atypical NLS. Using GFP constructs, the wild-type ARTS1 protein was shown to be localized both in the nucleus and in the cytoplasm. The mutant ARTS1 AC-terminus protein has the same intracellular protein (FIG. 6). ARTS1 may be involved in novel cytoplasmic/nuclear membrane trafficking and/or signaling cascades that are important in different types of cells.

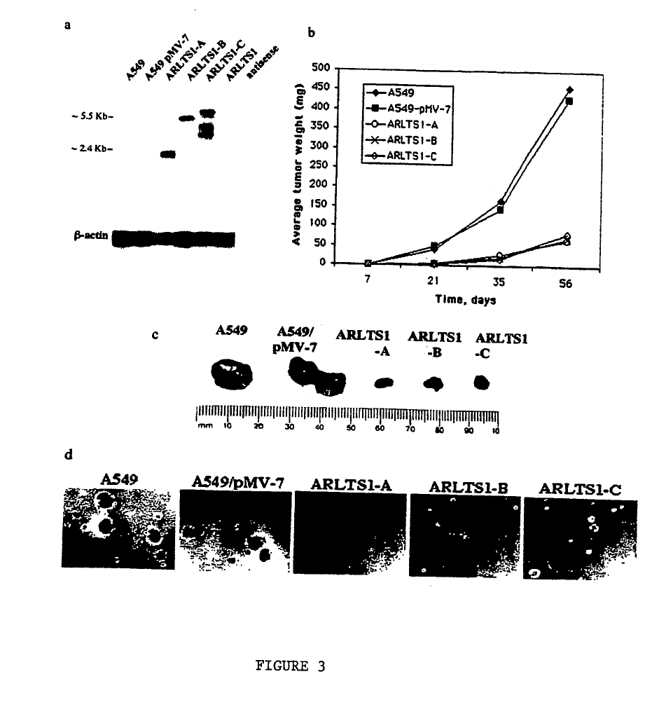

Northern and RT-PCR expression data showed that ARTS1 expression was dramatically decreased in A549, a highly tumorigenic non-small cell lung carcinoma (NSCLC) cell line (Fogh, J., Fogh, J. M. & Orfeo, T. One hundred and twenty-seven cultured human tumor cell lines producing tumors in nude mice. J Natl Cancer Inst 59, 221-6 (1977) which is incorporated herein by reference) when compared to the level found in normal lung. The ARTS1 ORF under the control of the LTR promoter was transfected into A549. Several stable clones were obtained and five of them were used in experiments: parental A549, the A549-pMV-7 (empty vector) clone, and three neomycin-resistant transfectants, selected according to the level of expression of the transfected ARTS1 minigene (FIG. 3). To evaluate the biological effect of ARTS1 in vitro and in vivo, tumorigenicity was examined by soft agar and in Nu/Nu nude mice (FIG. 3). All three transfected clones give rise to smaller colonies with a shorter survival in comparison with the parental cell or cells transfected with the empty vector. Furthermore, during 10 weeks of observation after the s.c. injection, the former consistently formed smaller, nonprogressive tumors, while the latter formed large, progressively growing tumors in nude mice. Thus, ARTS1 by itself has significant tumor-suppressor activity in A549 cells.

The presence of a new tumor suppressor within the well-characterized superfamily of Ras oncogenes is not as contradictory. It was recently shown that wild type Kras2 could inhibit lung carcinogenesis in mice, clearly illustrating the tumor suppressor role of the gene in lung tumorigenesis (Zhang, Z. et al. Wildtype Kras2 can inhibit lung carcinogenesis in mice. Nat genet 29. 25-33. (2001) which is incorporated herein by reference). The principal mechanism for ARTS1 inactivation in human cancers is biallelic methylation, as was proposed in the revised Knudson's two hit hypotheses (Jones, P. A. & Laird, P. W. Cancer epigenetices comes of age. Nat Genet 21, 163-7. (1999) which is incorporated herein by reference). One intriguing aspect of ARTS1 involvement in human cancer is the real significance of the G446A (Trp149Stop) nonsense mutation. Because the frequency of G446A mutation is about three times higher in familial cancers as in the general population and about two times higher as in sporadic cancers, one possible explanation is that ARTS1 germline mutations have low penetrance and are associated with a small percentage of familial melanoma or familial CLL cancers (which harbor a ten times higher frequency of the truncating mutation as in the same population control group). According to this, it is possible that there exists kindreds which carry the mutation but did not develop cancer. The same is true also for some other TSGs as is the case of BRCA2 germline mutations in breast and pancreatic cancers (Goggins, M. et al. Germline BRCA2 gene mutations in patients with apparently sporadic pancreatic carcinomas. Cancer Res 56, 5360-4. (1996) which is incorporated herein by reference). An alternative explanation is that this truncating mutation does not have a pathogenetic role in human cancers, because the lost domains are not important for tumorigenesis or because the protein has redundant functions with other ARL family members. Until now, only one polymorphic stop codon was identified in cancer related genes, the Lys3326ter in BRCA2 gene (Mazoyer, S. et al. A polymorphic stop condon in BRCA2. Nat Genet 14, 253-4. (1996) which is incorporated herein by reference). However, until independent groups analyze a larger number of cases, the possibility that such polymorphisms are associated with a modest increased cancer risk or are associated with other phenotypes in the heterozygous or homozygous state cannot be excluded.

Methods

Cell Lines. Eighty cell lines derived from human tumors were used in this study. Forty-four were hematopietic cancer cell lines and 36 were solid tumors cell lines (for detailed list, see Table 3). As controls, six lymphoblastoid cell lines made from peripheral blood lymphocytes of patients with Alzheimer's disease by transformation with Epstein Barr Virus (EBV) were used. All the cell lines were obtained from the American Type Culture Collection (ATCC) (Manassas, Va.) and maintained according to ATCC instructions.

Patient Samples. Experimental samples were derived from sporadic tumors or from peripheral blood of patients with familial cancer (total of 325). Control samples were derived from blood of patients with diseases other than cancer or from healthy individuals (total of 475). All samples were obtained with informed consent following institutional guidelines for the protection of human subjects. The 216 human sporadic tumors analyzed include 65 thyroid tumors, 58 colorectal adenocarcinomas, 48 breast carcinomas, 39 B-CLLs, 5 lung carcinomas and 1 idiopathyc pancytopenia. The panels of DNA from blood include: a) 69 DNA samples from females with BRCA-1- and BRCA-2-negative familial breast cancer; b) 17 DNA samples from males affected with prostate cancer and malignant melanoma which had been found negative for mutations at the p16 locus; c) 17 DNAs from patients with familial CLL (at least two first-degree relatives affected) and d) 6 DNAs from individuals with pancreatic cancer or melanoma who have a family history of at least one case of melanoma or pancreatic cancer and negative for mutations in the p16 and p14 genes. Patients' profile was similar for both groups: about 60% of cancer patients were from European Caucasian origin and the remaining 40% were from US persons. In the control group the proportions of the two cohorts were 75% and 25%, respectively. No bias toward distinct population groups (such as Ashkenazim) was noted. High molecular weight (HMW) DNA was extracted by conventional protocols (Sambrook, J., Frisch, E. F. & Maniatis, T. Molecular cloning: A Laboratory Manual, (Cold Spring Harbor Laboratory Press, Cold Spring Harbor, N.Y., 1989). which is incorporated herein by reference).

Rapid Amplification of cDNA Ends (RACE). The 3′ and 5′ ends of mRNAs were obtained by RACE from human testis, fetal liver, bone marrow and lymph node, using the Marathon-ready and the SMART RACE protocols (Clontech, Palo Alto, Calif.). The PCR products were separated on 1.0-2.0% agarose gels and gel purified using the QIAquick gel extraction kit (QIAGEN) or cloned in TA vector using TOPO TA Cloning (Invitrogen Carlsbad, Calif.) and sequenced.

Northern blot analysis. Human multiple tissues Northern blots were purchased from Clontech and total RNA was extracted from tumor cell lines or tumors by the QIAGEN RNeasy mini kit (QIAGEN) according to the manufacturer's protocol. The membranes were hybridized with a 443-bp probe containing the majority of the ARTS1 open reading frame (ORF) labeled with ³²P dCTP by random priming (Prime-it II Kit, Stratagene). Prehybridization and hybridization were carried out in Church Buffer (7% SDS, 0.5M phosphate buffer pH 7.2, 10 mM EDTA) for 18-20 h at 65° C. as described in Sambrook SUPRA.

Reversed Transcription PCR (RT-PCR) analysis. The DNA sequence was confirmed by RT-PCR and a semiquantitative RT-PCR was performed to analyze the levels of gene expression in different normal and tumor tissues. Five microliters of cDNA were used for each PCR with Advantage2 PCR kit (Clontech) and 10 pmol of each gene-specific primer for 35 cycles of 94° C. for 20 s, 65° C. for 30 s, 68° C. for 1 min (for a complete list of primers used in this study, see Table 4). To ensure that the RNA was of sufficient purity for RT-PCR, a PCR assay with primers specific for GAPDH cDNA (Clontech) was used. Semiquantitative PCR was performed with 23 cycles of amplification for ARTS1 gene and 18 cycles for GAPDH, in order to remain within a range of linear increase in the amount of PCR product. RT-PCR products were separated by agarose gel electrophoresis and blotted on Hybond N+ nylon membranes following standard procedures in Sambrook SUPRA. Membranes were hybridized with the same probe and in the same conditions as for Northern blotting. The relative intensity of hybridization signals was analyzed with a PhosphoImager system (Molecular Dynamics).

Methylation analysis by Southern blotting. In order to identify the global level of methylation for the ARTS1 locus, five micrograms of total genomic DNA were digested with Bg1II alone or in combination with methylation-sensitive HpaII (Roche) using a total of 40 U of enzyme for 12 h. Digests were electrophoresed on 0.8% agarose gels and blotted on Hybond N+ positively charged nylon membranes (Amersham Pharmacia Biotech) and hybridized with the same ORF probe as described before.

Methylation-specific PCR. To analyze methylation levels in the 5′ upstream region of ARTS1, a region upstream of the first exon on ARTS1 was amplified and bisulfite sequencing was carried out as described in Frommer, M. et al. A genomic sequencing protocol that yields and positive display of 5-methylcytosine residues in individual DNA strands. Proc natl Acad Sci USA 89, 1827-31. (1992), which is incorporated herein by reference. Modified DNA (200 ng) was subjected to PCR PCR products were purified and directly sequenced in order to obtain average methylation levels. In addition, PCR products were subcloned and at least six clones were sequenced to confirm direct sequencing data. Because of the unavoidable contamination of normal cells in the tumor specimens, we defined a CpG site as “hypermethylated” when more than 70% of PCR products contained bisulfite-resistant cytosines “Partial methylation” indicates detection of these products in 20-70% of the total products.

LOH studies. The paired normal and colorectal tumor DNA samples were tested for LOH by PCR amplification with oligonucleotide primers for microsatellite markers at D13S165 and D13S273 using fluorescent-labeled primers (ABI). One single nucleotide polymorphism found inside the ORF of ARTS1 (T442C) was heterozygous in about 45% of sequenced samples and was very useful for the rapid discrimination of informative/noninformative patients. The amplification products were run on an Applied Biosystems Model 377 DNA sequencing system (PE, Applied Biosystems). The LOH data for 39 paired normal/tumor B-CLL samples used in this study were previously reported in Bullrich, F. et al. Minimal region of loss at 3q14 in B-cell chronic lymphocytic leukemia. Blood 88, 3109-15 (1996), which is incorporated herein by reference.

Mutation detection. Primers used in mutation analysis were designed from intronic sequences directly upstream of the second exon and within the 3′ UTR region of ARTS1. PCRs were carried out for 35 cycles of 94° C. for 30 s, 62° C. for 30 s and 72° C. for 1 min using RedTaq genomic DNA polymerase (Sigma-Aldrich, St. Louis, Mo.), purified with the QIAquick PCR purification kit (QIAGEN) and then both strands were directly sequenced using the Applied Biosystems Model 377 DNA sequencing system (PE, Applied Biosystems, Foster City, Calif.). The 203 normal controls from the Italian population were analyzed by denatured high-performance liquid chromatography (DHPLC) (Transgenomics, Omaha Nebr.). The temperature used for heteroduplex formation was 57° C. and all the samples with abnormal patterns were directly sequenced.

Subcellular localization. The pEGFP N1-ARTS1 vector was prepared by digesting pEGFP N1 (Clontech) with SmaI; the insert was obtained by amplifying the ARTS1 full-length insert with Pfu where its stop codon was eliminated in order to generate an ARTS1-EGFP protein fused at the C-terminus. An additional pEGFP N1-ARTS1 ÄC-terminus vector was prepared carrying the ARTS1 protein truncated at position 446 of the ORF where the stop mutation is located. 293 cells were transfected by calcium phosphate (ProFection from Promega, Madison Wis.) and cultured on a cover slip and 24-48 h after transfection cells were analyzed by fluorescence microscopy as described in Ghosh, K. & Ghosh, H. P. Role of the membrane anchoring and cytoplasmic domains in intracellular transport and localization of viral glycoprotiens. Biochem Cell Biol 77, 165-78 (1999), which is incorporated herein by reference.

Stable transfection of A549 cells. A549 cell line was cultured in RPMI supplemented with 10% fetal bovine serum for the following studies. ARTS1 expression vector p-MV7-ARLTS1-sense was constructed by ligating the ARTS1 open reading frame in sense orientation into a mammalian expression vector pMV-7. All constructs were sequenced in order to exclude random mutants and were transfected by FuGENE6 transfection reagent according to the protocol (Boehringer Mannhiem). Transfected cells were selected with G418.

Analysis of transformed phenotype. Soft-agar colony assay of A549 wild-type and ARTS1 stable transfectants were performed as described in Trapasso, F. et al. Rat protein tyrosine phosphatase eta suppresses the neoplastic phenotype of retrovirally transformed thyroid cells through the stabilization of p27 (Kip1). Mol Cell Biol 20, 9236-46. (2000) which is incorporated herein by reference. A suspension of 106 cells in PBS (0.2 ml) was injected subcutaneousy into the right flank of Nu/Nu athymic mice (Jackson Laboratories Charles River, Cambridge, Mass.). Mice were sacrificed after 1, 3, 5, and 8 weeks and tumors were removed, weighed and measured in three dimesions. All experiments were perfomed in accordance with institutional guidelines.

Statistical analysis. Statistical analysis of results was performed using the Fisher's exact test; a P value of <0.05 was considered statistically significant. The cancer risk associated with the specific mutations identified in this study was anayzed using the odds ration (OR).

TABLE 1
ARTS1 sequence analysis in human cell lines, tumors and normal controls.

| | Amino | Amino acid | | | Familial | Normals, |
|---|---|---|---|---|---|---|
| Variant | acid | conservation | Cell Lines | Sporadic | cancers, | blood |
| name¹ | Change | (%)² | (%) | tumors (%) | blood (%) | (%) |
| T50 to C | Met 17 | 9/14 (65) | 2/80 (2.5)³ | 0/216 | 0/109 | 0/272 |
| | to Thr | | | | | |
| C65 to T | Ser 22 | 3/14 (21), PM1 | 0/80 | 2/216(1)⁴ | 0/109 | 1/272 (0.4) |
| | to Leu | site | | | | |
| C262 to A | Leu 88 | Leu only in | 1/80 (1)³ | 0/216 | 0/109 | 0/272 |
| | to Met | ARTS1 | | | | |
| C392 to T | Pro | 4/14 (29) | 6/80 (7.5) | 14/216 (6.5) | 4/109 (4) | 17/272 (6.25) |
| | 131 to | | | | | |
| | Leu | | | | | |
| T442 to C | Cys | Cys only in | 25/80 (31) | 127/216 (59) | 80/109 (73) | 182/272 (67) |
| | 148 to | ARTS1 | | | | |
| | Arg | | | | | |
| G446 to A | Trp | 12/14 (86) | 2/80 (2.5) | 8/216 (3.7) | 6/109 (6) | 10/475 (2.1) |
| | 149 to | | | | | |
| | Stop | | | | | |
| G490 to A | Glu | 9/14 (65) | 0/80 | 1/216 (0.5)⁴ | 0/109 | 0/272 |
| | 164 to | | | | | |
| | Lys | | | | | |

Note:
¹We identified also several synonymous polymorphisms such as: C175 to T (Leu 59); G 297 to A (Ser 99); C345 to T (Val 115); G396 to C (Leu 132); G546 to A (Gln 182).
²Data obtained by a multiple alignment of ARTS1 protein with ARF1 to ARF6 and ARL1 to ARL7 at the GenomeNet CLUSTALW server.
³Found only in melanoma cell lines.
⁴Found only in thyroid adenomas.

TABLE 2
Clinical data from families with G446A (Trp149Stop) mutation.

| Proband, sex, age¹ | Cancer type | Cancer Family history |
|---|---|---|
| KRR0003, female, 46 | B-CLL | Twin sister G446A +ve with B-CLL |
| TOR-1B, male, 57 | B-CLL and lung | Sister, 53, homozygous G446A with Thyroid |
| | cancer | adenoma; his son, 30, obligate carrier, |
| | | Essential thrombocytemia |
| | | Brother, heterozygous G446A - normal |
| | | Mother, dead, obligate carrier, B-CLL at |
| | | 80 yrs-old |
| | | Father, 86, obligate carrier, B-CLL |
| P/M 35003, male, ? | Gastric, 72 | None |
| | Melanoma, 72 | |
| | Prostate, 73 | |
| P/M 35012, male, ? | Prostate, 66 | Mother, cancer, unknown location, ? |
| | Melanoma, 67 | Brother, prostate, 73 |
| | | Sister, “black moles”, ? |
| | | Daughter, breast, ? |
| 1054-22671, male, | Melanoma, 50 | Paternal uncle, melanoma, ? |
| dead | Lung metastasis, 55 | Paternal aunt, pancreatic, ? |
| | | Paternal cousin, pancreatic, ? |
| | | Paternal cousin, head and neck, ? |
| 15-265-S87, female, ? | Bilateral breast | Daughter, 48, G446A carrier, unaffected |
| | cancer, 32 and 35 | |
| | Ovarian cancer, 50 | |

Note:
¹? = Age data unknown

TABLE 3
Cell lines used in the described experiments

| | |
|---|---|
| Hystotype | Cell lines used |

| | |
|---|---|
| Burkitt's lymphoma | AG876, AS283, BL2, BL30, BL41, CA46, |
| | DA978, Daudi, EB-B, ED36, Jiyoye, Lauckes, |
| | Nanalwa, P3HR-1, Raji, Ramos, RS11864, |
| | SKDHL and WMN |
| Multiple myeloma | HuNS1, MC/CAR, NC1-H929, RPM18226 and |
| | U266B1 |
| Large cell lymphoma | DB and SR |
| Immunoblastic | JM1 |
| B cell lymphoma | |
| Diffuse mix lymphoma | HT |
| Hodgkin's | RPM16666 and Hs445 |
| disease | |
| Non-Hodgkin's | RL |
| disease | |
| B-ALL | MV4; 11, RS4; 11, 697 |
| T-cell lymphomas | CEM, Del 1, HH, HSB2, HuT 102, MOLT-3, |
| and leukemias | MOLT-4, and MJ |
| Hairy cell leukemia | Mo T |
| CML-Erythroid | K562 |
| leukemia | |
| Lung carcinomas | A549, AFL, Calu-3, H69, H460, H1299, |
| | SKMES, 498 and 1285 |
| Pancreatic carcinomas | AsPC1, BxPC3, Capan-2, CFPAC-1, HS766T, |
| | MiaPaca, PANC1, PSN1, and SU8686 |
| Esophageal cancers | TE1, TE2, E10, TE15, KY200 and KY300 |
| Malignant melanoma | M14, 1007 MP, IR 6, WM 266.4, 397 MEL, |
| | 13443 and four cell lines derived from |
| | melanoma patients |
| Colon carcinoma | LoVo |
| Cervical carcinoma | HeLa |

TABLE 4
Primers used in the described experiments

| | | |
|---|---|---|
| Primer name | Primer sequence (5′-3′) | Application |

| | | | |
|---|---|---|---|
| 3′-ex2F | 5′ - CCA TGG GTT CTG TGA ATT | (SEQ ID NO:5) | Northern blot analysis |
| | CCA GAG G | | |
| 5′-ex2R2 | 5′ - CAG TGG TCC TGG AAT CTC | (SEQ ID NO:6) | |
| | TCT AGA C | | |
| 3′ex1F | 5′ - GCC AGC AGA AAG CAG CTC | (SEQ ID NO:7) | Reversed Transcription PCT |
| | CAT AGG | | (RT-PCR) analysis |
| 5′ex2R1 | 5′ - TTC AGG AGG CTC CAC AGG | (SEQ ID NO:8) | |
| | CTC TGC | | |
| MET-F | 5′ - GAG GTA TGT ATT GAA AG | (SEQ ID NO:9) | Methylation-specific PCR |
| | AAG AGG | | |
| MET-R | 5′ - AAC AAA ACC CAA TAA CAA | (SEQ ID NO:10) | |
| | CTC CA | | |
| ORF-F1 | 5′ - CAG AAG ACA GTA GCT GAT | (SEQ ID NO:11) | Genomic Mutation detection |
| | GTG | | |
| ORF-R2 | 5′ - GAG CAA AGA TAT GCT GCT | (SEQ ID NO:12) | |
| | CTG | | |
| MaeI-F1 | 5′ - GCT GAG TCC AGA GAG ATT | (SEQ ID NO:13) | G446A (Trp149Stop) |
| | CCA GG | | detection by MaeI digestion |
| MaeI-R1 | 5′ - TCT CGC CTG CAG ACA CAT | (SEQ ID NO:14) | |
| | GC | | |

TABLE 5
Expression levels and methylation status of the ARLTSI promoter in human
cancer cell lines

| Name | Origin | ARTSI expression^{a} | Methylation |
|---|---|---|---|
| Normal lung 1 | Normal lung | + | Low |
| Normal lung 2 | Normal lung | + | Low |
| A 549 | Lung carcinoma | □ | Hypermethylation |
| AFL | Lung carcinoma | □/+ | Hypermethylation |
| Calu-3 | Lung carcinoma | □/+ | ND |
| H 1299 | Lung carcinoma | □ | Hypermethylation |
| H 69 | Lung carcinoma | □/+ | ND |
| 1285 | Lung carcinoma | □/+ | ND |
| H 460 | Lung carcinoma | □/+ | Hypermethylation |
| Lymphoblastoid 1 | Immortalized lymphoblasts | + | Low |
| Lymphoblastoid 2 | Immortalized lymphoblasts | + | Low |
| Del 1 | T cell lymphoma | □/+ | Hypermethylation |
| HH | T cell lymphoma | □ | Hypermethylation |
| HSB 2 | T cell ALL | □ | Hypermethylation |
| HuT 102 | T cell lymphoma | □ | Hypermethylation |
| K 562 | CML-Erythroid leukemia | □ | ND |
| MJ | T cell lymphoma | □ | Hypermethylation |
| Mo T | T cell lymphoma | □ | Hypermethylation |
| AS 283 | Burkitt's lymphoma | + | Low |
| BL 41 | Burkitt's lymphoma | + | Low |
| PSN 1 | Pancreatic carcinoma | □/+ | Hypermethylation |
| MiaPaca | Pancreatic carcinoma | □/+ | Hypermethylation |
| HeLa | Cervical carcinoma | □ | Hypermethylation |
| SW 480 | Colon carcinoma | □ | ND |
| G-361 | Melanoma | □ | ND |

^{a}+, normal expression; +/−, reduced expression and −, absent expression;
ND—not done

TABLE 6
Allele frequency of G446A (Trp149Stop) in unrelated cancer patients
and control cases.

| Cancer patients | | | | Normal controls | | |
|---|---|---|---|---|---|---|
| | | Sample | | | Sample | |
| | | size, | | | size, | |
| Tumor type | Source | origin | G446A | Source | origin | G446A |
| Colorectal cancers | Bucharest, | 58, | 2 | Philadelphia | 116, | 1 |
| “sporadic” | Romania | tumor | | | blood | |
| Breast “sporadic” | Ferrara, | 38, | 3 | Bucharest | 156, | 2 |
| | Italy | tumor | | | blood | |
| Breast “sporadic” | Aarhus, | 10, | 0 | Ferrara | 203, | 7 |
| | Denmark | tumor | | | blood | |
| CLL “sporadic” | US | 39, | 0 | | | |
| | | tumor | | | | |
| Lung “sporadic” | Milan, | 5, tumor | 1 | | | |
| | Italy | | | | | |
| Thyroid “sporadic” | Catanzaro, | 65, | 1 | | | |
| | Italy | tumor | | | | |
| CLL familial | Paris, | 11, | 1 | | | |
| | France | blood | | | | |
| CLL familial | US | 6, blood | 1 | | | |
| Breat familial | Philadelphia, | 69, | 1 | | | |
| | PA | blood | | | | |
| Melanoma + prostate | Philadelphia, | 17, | 2 | | | |
| | PA | blood | | | | |
| Pancreatic + melanoma | Philadelphia, | 6, blood | 1 | | | |
| | PA | | | | | |
| Idiopathyc | Bucharest, | 1, blood | 1 | | | |
| Pancytopenia | Romania | | | | | |
| Total | | 325 | 14 | | 475 | 10 |
| | | | (4.30%) | | | (2.10%) |

What is claimed is:

1. An isolated protein comprising the amino acid sequence of SEQ ID NO:2.

2. An isolated nucleic acid molecule that comprises a nucleic acid sequence that encodes the protein of claim 1.

3. An isolated nucleic acid molecule comprising SEQ ID NO:1 or a fragment thereof having at least 10 nucleotides.

4. The nucleic acid molecule of claim 3 consisting of SEQ ID NO:1.

5. A recombinant expression vector comprising the nucleic acid molecule of claim 3.

6. A host cell comprising the recombinant expression vector of claim 5.

7. The nucleic acid molecule of claim 3 consisting of a fragment of SEQ ID NO:1 having at least 10 nucleotides.

8. The nucleic acid molecule of claim 3 consisting of a fragment of SEQ ID NO:1 having 12-150 nucleotides.

9. The nucleic acid molecule of claim 3 consisting of a fragment of SEQ ID NO:1 having 15-50 nucleotides.

10. An oligonucleotide molecule comprising a nucleotide sequence complementary to a nucleotide sequence of at least 5 nucleotides of SEQ ID NO:1.

11. The oligonucleotide molecule of claim 10 wherein said oligonucleotide molecule comprises a nucleotide sequence complementary to a nucleotide sequence of 5-50 nucleotides of SEQ ID NO:1.

12. The oligonucleotide molecule of claim 10 wherein said oligonucleotide molecule comprises a nucleotide sequence complementary to a nucleotide sequence of 10-40 nucleotides of SEQ ID NO:1.

13. The oligonucleotide molecule of claim 10 consisting of a nucleotide sequence complementary to a nucleotide sequence of at least 10-150 nucleotides of SEQ ID NO:1.

14. The oligonucleotide molecule of claim 10 consisting of a nucleotide sequence complementary to a nucleotide sequence of at least 18-28 nucleotides of SEQ ID NO:1.

15. An isolated antibody which binds to an epitope on SEQ ID NO:2.

16. The antibody of claim 15 wherein said antibody is a monoclonal antibody.

17. A method of identifying modulators of Caspase-1 protein protease activity comprising the steps of:
performing a test assay by contacting a Caspase-1 protease protein with a Caspase-1 substrate in the presence of a test compound,
determining the level of processing of said substrate by said protease, and
comparing said level to the level of processing of a Caspase-1 substrate by Caspase-1 protease protein in the absence of said test compound.

18. The method of claim 17 wherein said protein has SEQ ID NO:2.

19. The method of claim 17 wherein said substrate is FKBP46 protein.


SEQUENCE LISTING (17 sequences)

SEQ ID NO: 1 - DNA, 3791 bp, Homo sapiens
cacccaagtctgagttgctaaaaaatggagctgtcactgggccttgctctgccaggacctgcagagccggggacctctctgtggcaagcccagcaagatgactgctctgaggcgccctagggctgagggaggggccgtgacaccagccccgccccccagccacctgggaaaaggaagcacaaaaaggagaagcagcaacggctgctctgcttccttcccatctcgctcttgggtcatgcctggccagcagaaagcagctccataggggaggagagccacgcaggatctcacagctgcagtctaatagtaacacagaggattcagcagtggccaccatgggttctgtgaattccagaggtcacaaggcggaagcccaggtggtgatgatgggcctggactcggcgggcaagaccacgctcctttacaagctgaagggccaccagctggtggagaccctgcccactgttggtttcaacgtggagcctctgaaagctcctgggcacgtgtcactgactctctgggacgttggggggcaggccccgctcagagccagctggaaggactatctggaaggcacagatatcctcgtgtacgtgctggacagcacagatgaagcccgcttacccgagtcggcggctgagctcacagaagtcctgaacgaccccaacatggctggcgtccccttcttggtgctggccaacaagcaggaggcacctgatgcacttccgctgcttaagatcagaaacaggctgagtctagagagattccaggaccactgctgggagctccggggctgcagtgccctcactggggaggggctgcccgaggccctgcagagcctgtggagcctcctgaaatctcgcagctgcatgtgtctgcaggcgagagcccatggggctgagcgcggagacagcaagagatcttgatccagacagagcagcatatctttgctcatacaaactagaagaaccagctgatccttgagaaatttacgcttagtctatcaaacaagaaatgctggcttggcccggtggctcatgcctgtaatcccagcactgtgggagaccacggtgggggaatcccttgagcccaggagttggagagcaacatcacaacaccccatttctactaataatcaaaaaattggccgggcatggtggcatgtgcctgtagtcccagctacttgggaggctgaggcaggagaatcgcttgagcccaagaggtagaggttgcagtgagccaagatcgcgccactgcactccagtctgggcaacagagtgagaccctgtctcaataataataataataataatgatgatactctaagaaaaaaatctcaacatacttcatttaatagctcgttaccaagtgtgaatgaagcaatatgtcataatagagtagccactggttgcataataatagagacctaaattctcaaatagggaaagaggttttaaaatcaaatttgaggccaggtgcagtggctcatgggcggaggagggcagattacttgaggctaggagttcaagaccagcctggccaacatggtgaaaccccatctctactgaaaatacaaaaattaggcatagtggtgcacgcctgcagtcccagctactcaggaggttgaggcagaagaatcgcttgaacccaggaagtggaggttgcagtgagccgagattgtgctgctgcactccagcctgggtgaaaaagacaggctgtgtctccaaaaagaaaaaaaaaagtcaaattcaaatatcatctggacatgtcacaatggatcgcggatccttatgagtgattttccccagtggcccctggggatgtgccactgtcactcagaagggcaagctaggcagggcccatccaacagcaggggtctgcaggttagacgttccctgccctgggacgctcacccctgggcaagaggctggaagttcacaccatccaaaatttatccttgttttttttctgatgctaattagcctctcccgattttatgacatcttgtgttgatctttttcaaaaactcattttcttttttttccttctcttttctccttcttgtagcacatatctttcgttaaagatcagatcaataaaatattttatttattcattaatttaacaaaaaaaacagagcatttagtttgtggcaaaaacactgagctttcgaatatgaatcatgtgctttaggtgggagttgtgaattctgaagatacagatgacagtgacgaatgccttctgtctcatgattgacagggaaaaggaaggttgaccatagcatcctagaaggctcatcaggtgatcattacctagcatccatgaagcacctgaaattatttgcaaaatgttacgctttggaccatttttccggggaaggagatccagaactttttaccagattttcaaagacatctgtgactcccaaaagttaacaatcactgatgtggttgttgtatccctcatccaaccccagaacactttctgtaatctgagttttttaatggcaagtggcctatatttagcacctgttctcatgttaaacagctctgaatgttagatattctttcttatcctggactggttctctctatctctggagtaatgcagtataaattggccatcagtaccctcctaaaatctgagatctgccaggcccctcttctaacaccaggttaggcatgcttggttatttccagtacttgtgagtcaacatgtttcaagacgctgtgttagacactagggatgcaaagatgaatgagataaggcctcaggcctcatggaaggtgagacagtaaagacattactcccataaaaatgtgaggagagagactcagttcagcaactgtttattctgtttattgagcacttacttggaccaagcactgtggtcttggtgttttacatagactgtctctaattctcacaactctgcaaaatatatatattcccattttataaaactacaaactgaggctcagagaaggtgtgacctcttgttgcttgaggcacagagttataaagtaacatatctggaatttgaaatgagatctgtttagggctaatgctgcatttttctacaacatcatgcctctagaaggtttaagctaggtaggctttcagccagcagacatgatggggagagccttctaataagagggaagagactgcttggaagcatgaagggaggtgtaagaaagataagtaagtcagtgtacttgcaacagaggcttgggatgaagggtgggtgaagttgacatcacgatagaaaacaaaactggaatgggagtttaggtccaatttgggcaaggttgtttgaatttcaataatcaggggtttgggtcaaggaagaaaaatcatgggacttgccatttaggaggataattttgtggtagtgtggaggtgaaataaagagaaaagggaaccttggagctgggaaggcaggaaaccggctagatgaccatcacacagcaaaggagggagtggaagagagatgagaaaattgagagctattattaagaaaaacagttgagagaggaagaatttgaagagggctcaagattttgagtccacatgacagaaggactggaatgccatgaactggagaaggtgagcgctgaagaaccaggatgggacggggctggaacagctgggttcagcttttgcagggtgggtacgtgtttggttatagctgctttcagattgttccattatctgtactcccaacaaccctgccggatatatttgttggctttcactcaaaaaaaaaaaaaaaaaaaaaaaaaaaaaaa

SEQ ID NO: 2 - protein, 196 aa, Homo sapiens
MGSVNSRGHKAEAQVVMMGLDSAGKTTLLYKLKGHQLVETLPTVGFNVEPLKAPGHVSLTLWDVGGQAPLRASWKDYLEGTDILVYVLDSTDEARLPESAAELTEVLNDPNMAGVPFLVLANKQEAPDALPLLKIRNRLSLERFQDHCWELRGCSALTGEGLPEALQSLWSLLKSRSCMCLQARAHGAERGDSKRS

SEQ ID NO: 3 - RNA, 24 nt, Artificial Sequence
  Ribozyme
cugaugaguccgcgaggacgaaac

SEQ ID NO: 4 - RNA, 26 nt, Artificial Sequence
  Ribozyme
ncugaugaguccgcgaggacgaaacn

SEQ ID NO: 5 - DNA, 25 bp, Artificial Sequence
  Oligonucleotide primer
ccatgggttctgtgaattccagagg

SEQ ID NO: 6 - DNA, 25 bp, Artificial Sequence
  Oligonucleotide primer
cagtggtcctggaatctctctagac

SEQ ID NO: 7 - DNA, 24 bp, Artificial Sequence
  Oligonucleotide primer
gccagcagaaagcagctccatagg

SEQ ID NO: 8 - DNA, 24 bp, Artificial Sequence
  Oligonucleotide primer
ttcaggaggctccacaggctctgc

SEQ ID NO: 9 - DNA, 23 bp, Artificial Sequence
  Oligonucleotide primer
gaggtatgtattgaaagaagagg

SEQ ID NO: 10 - DNA, 23 bp, Artificial Sequence
  Oligonucleotide primer
aacaaaacccaataacaactcca

SEQ ID NO: 11 - DNA, 21 bp, Artificial Sequence
  Oligonucleotide primer
cagaagacagtagctgatgtg

SEQ ID NO: 12 - DNA, 21 bp, Artificial Sequence
  Oligonucleotide primer
gagcaaagatatgctgctctg

SEQ ID NO: 13 - DNA, 23 bp, Artificial Sequence
  Oligonucleotide primer
gctgagtccagagagattccagg

SEQ ID NO: 14 - DNA, 20 bp, Artificial Sequence
  Oligonucleotide primer
tctcgcctgcagacacatgc

SEQ ID NO: 15 - DNA, 17 bp, Homo sapiens
agctcccagcagtggtc

SEQ ID NO: 16 - DNA, 17 bp, Homo sapiens
  misc_feature 7, 11: n = A,T,C or G
agctccnagcngtggtc

SEQ ID NO: 17 - DNA, 17 bp, Homo sapiens
agctcctagcagtggtc